(19) United States
(12) Patent Application Publication
(10) Pub. No.: US 2026/0000719 A1
(43) Pub. Date: Jan. 1, 2026

(54) METHOD OF INCREASING GLUCAGON-LIKE PEPTIDE-1 BY SYNBIOTIC COMPOSITION

(71) Applicant: Leeuwenhoek Laboratories Co. Ltd., Taipei City (TW)

(72) Inventors: Cheng-Chih HSU, Taipei City (TW); Chi-Shan LI, Taipei City (TW); Chiao-Wei LIN, Taipei City (TW); Rou-An CHEN, Taipei City (TW); Hsin-Yu LIAO, Taipei City (TW); Wen-Hui LIN, Taipei City (TW)

(21) Appl. No.: 19/251,755

(22) Filed: Jun. 26, 2025

Related U.S. Application Data
(60) Provisional application No. 63/666,664, filed on Jul. 1, 2024.

(30) Foreign Application Priority Data
Mar. 18, 2025 (TW) 114110122

(51) Int. Cl. A61K 35/745; A61K 31/198; A61K 35/00; A61K 36/77; A61P 3/04

(52) U.S. Cl. CPC A61K 35/745; A61K 31/198; A61K 36/77; A61P 3/04; A61K 2035/115

(57) ABSTRACT

A method of increasing glucagon-like peptide-1, including administering to a subject in need thereof an effective amount of synbiotic composition including lychee polyphenols, probiotics and methionine. Probiotics includes Bifidobacterium animalis BCRC910645, Bifidobacterium longum BCRC910812, or a combination thereof for increasing glucagon-like peptide-1. Also provided is a method of preventing or treating glucagon-like peptide-1 deficiency, including administering to a subject in need thereof an effective amount of synbiotic composition as above mentioned.

### CROSS-REFERENCE TO RELATED APPLICATION

This application claims priority to U.S. Provisional Application Ser. No. 63/666,664, filed on Jul. 1, 2024, and Taiwan Application Serial Number 114110122, filed on Mar. 18, 2025, the disclosures of which are incorporated herein by reference in their entireties.

The Sequence Listing associated with this application is filed in electronic format via EFS-Web and is hereby incorporated by reference into the specification in its entirety. The name of the XML file containing the Sequence Listing is NP-37530-US_SEQ_LIST.xml. The size of the XML file is 2,847 bytes, and the XML file was created on May 16, 2025.

### BACKGROUND

### Field of Invention

The present invention relates to a method of increasing glucagon-like peptide-1.

### Description of Related Art

Since 1975, the prevalence of obesity has almost tripled worldwide, mainly because of unhealthy eating habits. As obesity is associated with increased susceptibility to a variety of chronic diseases, the increasing prevalence of obesity has become an important public health issue worldwide. Although there are quite a few existing weight loss drugs, not every weight loss drug is effective for obese subjects. Since there are the many reasons causes of obesity, some obese subjects may be patients with glucagon-like peptide-1 (GLP-1) deficiency.

Therefore, how to provide a composition for preventing and treating obesity-related disease, the related art really needs to be improved.

### SUMMARY

The present disclosure provides a method of increasing glucagon-like peptide-1, comprising administering to a subject in need thereof an effective amount of synbiotic composition comprising: lychee polyphenols; probiotics, wherein the probiotics comprises Bifidobacterium animalis BCRC910645, Bifidobacterium longum BCRC910812, or a combination thereof; and methionine.

In some embodiments, the lychee polyphenols comprises proanthocyanidins, epicatechin, or a combination thereof.

In some embodiments, the synbiotic composition comprises: a weight percentage of the lychee polyphenols from 1% to 30%, a weight percentage of the probiotics from 10% to 50%, and a weight percentage of the methionine from 0.5% to 20%, based on a total weight of the synbiotic composition.

In some embodiments, the synbiotic composition further comprises indigestible maltodextrin, fructooligosaccharide, isomalto-oligosaccharide, probiotic fermented powder, rice bran concentrate, or a combination thereof.

In some embodiments, the probiotic fermented powder is prepared by the following: culturing a combination of Lactobacillus salivarius subsp. salicinius AP-32 strain, Lactobacillus plantarum LPL28 strain, Lactobacillus acidophilus TYCA06 strain, and Bifidobacterium longum subsp. infantis BLI-02 strain in a culture medium comprising milk, milk powder, casein, soybean, bean product, or whey for fermentation; after centrifuging the culture medium to obtain a fermentation liquid, removing strain activity of the fermentation liquid by heat sterilization, removing the strains in the fermentation liquid, and drying the fermentation liquid to form a dry powder; and mixing the dry powder and maltodextrin to obtain the probiotic fermented powder.

In some embodiments, the rice bran concentrate is obtained by following: mixing and grinding rice bran and water in a weight ratio of 1:1 to 1:10 to obtain a ground mixture; hydrolyzing the ground mixture with amylase to obtain a hydrolyzed mixture; and heating, centrifuging, and concentrating the hydrolyzed mixture to obtain the rice bran concentrate.

In some embodiments, the probiotics comprises the Bifidobacterium animalis BCRC910645 and the Bifidobacterium longum BCRC910812.

In some embodiments, the probiotics are live bacteria.

In some embodiments, an amount of the probiotics is from 1×10⁹ CFU/g to 1×10¹¹ CFU/g.

In some embodiments, the synbiotic composition is suitable for use in an obese subject.

In some embodiments, the obese subject is caused by following selected from the group consisting of diet, type 2 diabetes, hyperglycemia, glucose intolerance, dyslipidemia, insulin resistance, hyperinsulinemia, fatty liver, cardiovascular disease, stroke, cancer and combinations thereof.

In some embodiments, the synbiotic composition is a food composition or a pharmaceutical composition.

In some embodiments, the synbiotic composition is taken orally with a dose of from 1000 mg to 2400 mg per day.

In some embodiments, the synbiotic composition is taken orally with a dose of from 500 mg to 1200 mg twice per day.

In some embodiments, the synbiotic composition is administered orally or parenterally.

In some embodiments, the method further comprises increasing an amount of Akkermansia muciniphila (abbreviation as A. muciniphila or AKK) in intestine.

The present disclosure also provides a method of preventing or treating glucagon-like peptide-1 deficiency, comprising administering to a subject in need thereof an effective amount of synbiotic composition comprising: lychee polyphenols; probiotics, wherein the probiotics comprises Bifidobacterium animalis BCRC910645, Bifidobacterium longum BCRC910812, or a combination thereof; and methionine.

In some embodiments, the lychee polyphenols comprises proanthocyanidins, epicatechin, or a combination thereof.

In some embodiments, the synbiotic composition comprises: a weight percentage of the lychee polyphenols from 1% to 30%, a weight percentage of the probiotics from 10% to 50%, and a weight percentage of the methionine from 0.5% to 20%, based on a total weight of the synbiotic composition.

In some embodiments, the synbiotic composition further comprises indigestible maltodextrin, fructooligosaccharide, isomalto-oligosaccharide, probiotic fermented powder, rice bran concentrate, or a combination thereof.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 shows the Guifei Longum synbiotics (LE LIGHT) increase the amount of GLP-1 in plasma of obesity caused by a high-fat diet (HFD) according to some embodiments of the present disclosure. Data are mean values and standard deviations. Abbreviations: CON, control group; HF: high fat.

FIG. 2 shows the Guifei Longum synbiotics increase the amount of AKK bacteria in human body according to some embodiments of the present disclosure.

FIG. 3 shows the Guifei Longum synbiotics increase the amount of GLP-1 in human plasma according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number.

Since there are many reasons to induce obesity, some obese subjects may be GLP-1 deficiency patients. GLP-1 supplementation can slow down the peristalsis of the intestines, reduce the digestion rate, and thus reduce the speed of energy intake, achieving the ideal effect of weight loss.

The present disclosure provides a method of increasing glucagon-like peptide-1, including administering to a subject in need thereof an effective amount of synbiotic composition. The synbiotic composition including lychee polyphenols, probiotics, and methionine, in which the probiotics includes Bifidobacterium animalis BCRC910645, Bifidobacterium longum BCRC910812, or a combination thereof.

As used herein, the term “synbiotic” refers to nutritional foods or medicines that combine prebiotics and probiotics, and can also add postbiotic precursors to promote human health.

As used herein, the term “prebiotic” refers to substances that can be decomposed, utilized by probiotics, and promote the growth of probiotics, thereby producing beneficial effects on health.

As used herein, the term “postbiotic” refers to an inanimate microbial secretion or fragment product that provides physiological benefits to the host.

In some embodiments of the present disclosure, the main active ingredients of the synbiotic composition include lychee polyphenols, probiotics and methionine, in which the probiotics includes Bifidobacterium animalis BCRC910645, Bifidobacterium longum BCRC910812, or a combination thereof. B. animalis subsp. lactis CP-9 strain has been deposited in Bioresource Collection and Research Center of Taiwan Food Industry Research and Development Institute (address of depositary institution: No. 331 Shih-Pin Road, Hsinchu, Taiwan 300), the deposit number is BCRC910645, and the deposit date is Aug. 21, 2014, which can be purchased freely; or has been deposited in China Center for Type Culture Collection (address of depositary institution: No. 299, bayi road, Whuhan, Hubei, P.R.China), the deposit number is M2014588, and the deposit date is Nov. 24, 2014. B. longum subsp. infantis BLI-02 strain has been deposited in Bioresource Collection and Research Center of Taiwan Food Industry Research and Development Institute, the deposit number is BCRC910812, and the deposit date is Jan. 18, 2018, which can be purchased freely; or has been deposited in China General Microbiological Culture Collection Center (address of depositary institution: No. 1 West Beichen Road, Chaoyang District, Beijing 100101, China), the deposit number is CGMCC No. 15212, and the deposit date is Jan. 15, 2018.

In some examples, the probiotics includes Bifidobacterium animalis BCRC910645 and Bifidobacterium longum BCRC910812. In some examples, a weight ratio of the Bifidobacterium animalis BCRC910645 and the Bifidobacterium longum BCRC910812 is 1:0.1 to 1:10. In some examples, a weight ratio of the Bifidobacterium animalis BCRC910645 and the Bifidobacterium longum BCRC910812 is 1:2.

In some embodiments, the lychee polyphenols include proanthocyanidins, epicatechin, or a combination thereof. In some examples, proanthocyanidins include proanthocyanidins A1, A2, B1, B2, or a combination thereof. Catechins are the general term for flavanols in tea, including catechin (C), epicatechin (EC), epicatechingallate (ECG), epigallocatechingallate (EGCG), epicatechin-(4β→8, 2β→O→7) epicatechin-(4β→8)-eicatechin (A2-EC). In some examples, the lychee polyphenols are purchased from Oligonol® from Amino Up Co., Ltd. in Hokkaido, Japan, the main ingredient includes proanthocyanidins A1, A2, B1, B2, catechin, EC, ECG, EGCG, A2-EC, and green tea polyphenols.

In some embodiments, a weight percentage of the lychee polyphenols is from 1% to 30%, a weight percentage of the probiotics is from 10% to 50%, and a weight percentage of the methionine is from 0.5% to 20%, based on a total weight of the composition. Wherein the weight percentage of the lychee polyphenols is from 1% to 30%, for example, 2%, 3%, 4%, 5%, 6%, 7%, 8%, 9%, 10%, 11%, 12%, 13%, 14%, 15%, 16%, 17%, 18%, 19%, or any value between any two of these values. Wherein the weight percentage of the probiotics is from 10% to 50%, for example, 15%, 20%, 25%, 30%, 35%, 40%, 45%, or any value between any two of these values. Wherein the weight percentage of the methionine is from 0.5% to 20%, for example, 0.6%, 0.7%, 0.8%, 0.9%, 1%, 2%, 4%, 6%, 8%, 10%, 12%, 14%, 16%, 18%, or any value between any two of these values.

In some embodiments, the synbiotic composition further includes indigestible maltodextrin, fructooligosaccharide, isomalto-oligosaccharide, probiotic fermented powder, rice bran concentrate, green tea polyphenols, or a combination thereof as a nutritional supplement.

In some examples, probiotic fermented powder was purchased from Totipro® PE0401 probiotic fermented powder of Glac Biotech Co., Ltd. In detail, a preparation method of the probiotic fermented powder is herein incorporated by reference in its entirety in Taiwan patent number TW 1739495: a combination of lactic acid bacteria strains is cultured in a culture medium including milk, milk powder, casein, soybean, bean product, or whey for fermentation, the combination of lactic acid bacteria strains includes Lactobacillus salivarius subsp. salicinius AP-32 strain (BCRC 910437), Lactobacillus plantarum LPL28 strain (BCRC 910536), Lactobacillus acidophilus TYCA06 strain (BCRC 910813), and Bifidobacterium longum subsp. infantis BLI-02 strain (BCRC 910812). Next, after centrifuging the culture medium to obtain a fermentation liquid, strain activity of the fermentation liquid is removed by heat sterilization, the strains in the fermentation liquid are removed, and the fermentation liquid is dried to form a dry powder. Next, the dry powder and maltodextrin (for example, in a weight ratio of 2:3) are mixed to obtain the probiotic fermented powder.

In some examples, the rice bran concentrate is obtained by following: mixing and grinding rice bran and water in a weight ratio of 1:1 to 1:10 to obtain a ground mixture; hydrolyzing the ground mixture with amylase to obtain a hydrolyzed mixture; and heating, centrifuging, and concentrating the hydrolyzed mixture to obtain the rice bran concentrate. In some examples, rice bran and water are mixed in a weight ratio of 1:1, 1:2, 1:3, 1:4, 1:5, 1:6, 1:7, 1:8, 1:9, or 1:10.

In some examples, a weight percentage of the lychee polyphenols is from 1% to 30%, a weight percentage of the probiotics is from 10% to 50%, a weight percentage of the methionine is from 0.5% to 20%, a weight percentage of the indigestible maltodextrin is from 1% to 70%, a weight percentage of the fructooligosaccharide is from 1% to 50%, a weight percentage of the isomalto-oligosaccharide is from 0.5% to 50%, a weight percentage of the probiotic fermented powder is from 0.5% to 20%, a weight percentage of the rice bran concentrate is from 0.5% to 20%, and a weight percentage of the green tea polyphenols is from 1% to 50%, based on a total weight of the composition.

In some embodiments, the probiotics are live bacteria.

In some embodiments, an amount of the probiotics is from 1×10⁹ CFU/g to 1×10¹¹ CFU/g, for example, 2×10⁹ CFU/g, 3×10⁹ CFU/g, 4×10⁹ CFU/g, 5×10⁹ CFU/g, 6×10⁹ CFU/g, 7×10⁹ CFU/g, 8×10⁹ CFU/g, 9×10⁹ CFU/g, 1×10¹⁰ CFU/g, 2×10¹⁰ CFU/g, 3×10¹⁰ CFU/g, 4×10¹⁰ CFU/g, 5×10¹⁰ CFU/g, 6×10¹⁰ CFU/g, 7×10¹⁰ CFU/g, 8×10¹⁰ CFU/g, 9×10¹⁰ CFU/g, 1×10¹¹ CFU/g, or any value between any two of these values.

In some embodiments, the synbiotic composition is suitable for use in an obese subject. In some examples, the obese subject is caused by following selected from the group consisting of diet, type 2 diabetes, hyperglycemia, glucose intolerance, dyslipidemia, insulin resistance, hyperinsulinemia, fatty liver, cardiovascular disease, stroke, cancer and combinations thereof.

In some embodiments, the synbiotic composition is administered to a subject orally or parenterally. In some embodiments, the synbiotic composition is formulated into an oral dosage form selected from the group consisting of solution, suspension, emulsion, powder, lozenge, pill, syrup, buccal lozenge, tablet, chewing gum and capsule for administration to a subject.

In some embodiments, the synbiotic composition can be a pharmaceutical composition. In some examples, the pharmaceutical composition includes a pharmaceutically acceptable carrier including, but is not limited to water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

In some embodiments, the synbiotic composition can be a food composition. For example, added to edible materials in the form of food additives to prepare a food product for human or animal consumption. Food composition includes, but is not limited to, general foods, health foods, beverages, nutritional supplements, dairy products or feeds, etc. In some examples of oral dosage forms, the synbiotic composition may optionally include pharmaceutical and food-acceptable carriers, excipients and/or additives. In other examples, the dosage form of the synbiotic composition may include, but is not limited to, powders, tablets, granules, suppositories, microcapsules, ampoules, liquid sprays or suppositories.

In some embodiments, the synbiotic composition is taken orally with a dose of from 1000 mg to 2400 mg per day. In some examples, a dose of 1000 mg per day, a dose of 1100 mg per day, a dose of 1200 mg per day, a dose of 1300 mg per day, a dose of 1400 mg per day, a dose of 1500 mg per day, a dose of 1600 mg per day, a dose of 1700 mg per day, a dose of 1800 mg per day, a dose of 1900 mg per day, a dose of 2000 mg per day, a dose of 2100 mg per day, a dose of 2200 mg per day, a dose of 2300 mg per day, a dose of 2400 mg per day, or any value between any two of these values.

In some embodiments, the synbiotic composition is taken orally with a dose of from 500 mg to 1200 mg twice per day. In some examples, a dose of 500 mg twice per day, a dose of 550 mg twice per day, a dose of 600 mg twice per day, a dose of 650 mg twice per day, a dose of 700 mg twice per day, a dose of 750 mg twice per day, a dose of 800 mg twice per day, a dose of 850 mg twice per day, a dose of 900 mg twice per day, a dose of 950 mg twice per day, a dose of 1000 mg twice per day, a dose of 1050 mg twice per day, a dose of 1100 mg twice per day, a dose of 1150 mg twice per day, a dose of 1200 mg twice per day, or any value between any two of these values.

In some embodiments, the synbiotic composition is prepared as tablet or capsule, with a dosage of 500 mg/tablet or capsule to 2400 mg/tablet or capsule, for example, 500 mg/tablet or capsule, 600 mg/tablet or capsule, 700 mg/tablet or capsule, 800 mg/tablet or capsule, 900 mg/tablet or capsule, 1000 mg/tablet or capsule, 1100 mg/tablet or capsule, 1200 mg/tablet or capsule, 1300 mg/tablet or capsule, 1400 mg/tablet or capsule, 1500 mg/tablet or capsule, 1600 mg/tablet or capsule, 1700 mg/tablet or capsule, 1800 mg/tablet or capsule, 1900 mg/tablet or capsule, 2000 mg/tablet or capsule, 2100 mg/tablet or capsule, 2200 mg/tablet or capsule, 2300 mg/tablet or capsule, 2400 mg/tablet or capsule, or any value between any two of these values.

In some embodiments, the synbiotic composition is administered orally or parenterally.

In some embodiments, the method further includes increasing an amount of Akkermansia muciniphila in intestine. Akkermansia muciniphila is an extremely anaerobic Gram-negative bacterium and one of the most abundant species in the human intestinal microorganisms, accounting for about 1% to 5% of the total number of bacteria in the human intestinal tract. It decomposes mucin as the only source of carbon, nitrogen and energy, and produces short-chain fatty acids (SCFAs) such as acetate and propionate. Improving obesity is the first confirmed health benefit of Akkermansia muciniphila. Obesity is associated with chronic low-grade inflammation, which is related to specific changes in the gut microbiota. In obese individuals, there is an increase in the abundance of Firmicutes in the gut, while the abundance of Akkermansia muciniphila is significantly reduced. Additionally, the abundance of Akkermansia muciniphila is negatively correlated with body fat weight and impaired glucose tolerance. It has been found that Akkermansia muciniphila can also improve indicators related to metabolic. Akkermansia muciniphila can mitigate metabolic abnormalities induced by a high-fat diet, including increased fat mass, adipose tissue inflammation, and insulin resistance.

One embodiment of the present disclosure further provides a method of preventing or treating insufficient secretion of glucagon-like peptide-1 syndrome, comprising administering to a subject in need thereof an effective amount of synbiotic composition comprising: lychee polyphenols, probiotics and methionine as above mentioned.

One embodiment of the present disclosure further provides a method of preventing or treating reduced levels of glucagon-like peptide-1 syndrome, comprising administering to a subject in need thereof an effective amount of synbiotic composition comprising: lychee polyphenols, probiotics and methionine as above mentioned.

One embodiment of the present disclosure further provides a method of preventing or treating glucagon-like peptide-1 deficiency, comprising administering to a subject in need thereof an effective amount of synbiotic composition comprising: lychee polyphenols, probiotics and methionine as above mentioned.

A number of examples are provided herein to elaborate the composition of the instant disclosure. However, the examples are for demonstration purpose alone, and the instant disclosure is not limited thereto.

### EXAMPLE 1 SYNBIOTICS INCREASES GLP-1 SECRETION

The composition used in the examples was specifically Guifei Longum synbiotics (LE LIGHT, provided by Leeuwenhoek Laboratories Co. Ltd). The composition includes lychee polyphenols, probiotics, methionine, indigestible maltodextrin, fructooligosaccharide, isomalto-oligosaccharide, probiotic fermented powder, and rice bran concentrate. The amount of the lychee polyphenols was 10 wt %, the amount of the probiotics was 30 wt %, the amount of the methionine was 2 wt %, the amount of the indigestible maltodextrin was 30 wt %, the amount of the fructooligosaccharide was 10 wt %, the amount of the isomalto-oligosaccharide was 8%, the amount of the probiotic fermented powder was 6%, the amount of the rice bran concentrate was 4% (Rice bran and water were mixed and ground in a weight ratio of 1:5). The composition was prepared in the form of capsule, and the above weight percentages do not include the weight of the capsule.

The lychee polyphenols in the composition were purchased from commercial Oligonol®. Bifidobacterium animalis was selected from B. animalis subsp. lactis CP-9 strain (BCRC910645) and Bifidobacterium longum was selected from B. longum subsp. infantis BLI-02 (BCRC910812), in which the effective live bacteria included 1×10¹⁰ CFU/g. The probiotic fermented powder was purchased from Totipro® PE0401 probiotic fermented powder of Glac Biotech Co., Ltd.

In this example, 8-week-old C57BL/6 male mice were randomly divided into 3 experimental groups (n=12) and 1 control group (n=8). The experimental groups were given a high-fat diet to induce obesity; the control group was given a corresponding control diet. After the beginning of the experiment, the first experimental group was given a high-fat diet, and the second and third experimental groups were given a high-fat diet and were fed with two different doses (low dose: 150 mg/kg BW, high dose: 750 mg/kg BW) of Guifei Longum synbiotics (dissolved in PBS containing 2.5% glycerol) daily, respectively, with a tube feeding volume of 200 μL; the other groups of mice were fed with the same volume of PBS containing 2.5% glycerol daily, and the experimental period was 8weeks.

At the 8th week of the experiment, the concentrations of GLP-1 secreted by mice in different groups were tested and compared. After mice or humans ingest carbohydrates, fats, and proteins, intestinal L-cells will be induced to secrete GLP-1 into the blood. In order to test the difference in the efficacy of Guifei Longum synbiotics in promoting GLP-1 secretion in mice, glucose solution was given to mice after fasting, and the concentration of GLP-1 in the blood of mice after glucose ingestion was detected. The experimental process was as follows: after fasting for 5 hours, the mice were fed with glucose solution at a dose of 2 g/kg BW. Blood was collected 10 minutes later, and dipeptidyl peptidase-4 enzyme (DPP4) inhibitor was added to the blood sample to prevent GLP-1 from being degraded. After separating plasma from the blood sample, the amount of GLP-1 in the sample was analyzed using ELISA kit.

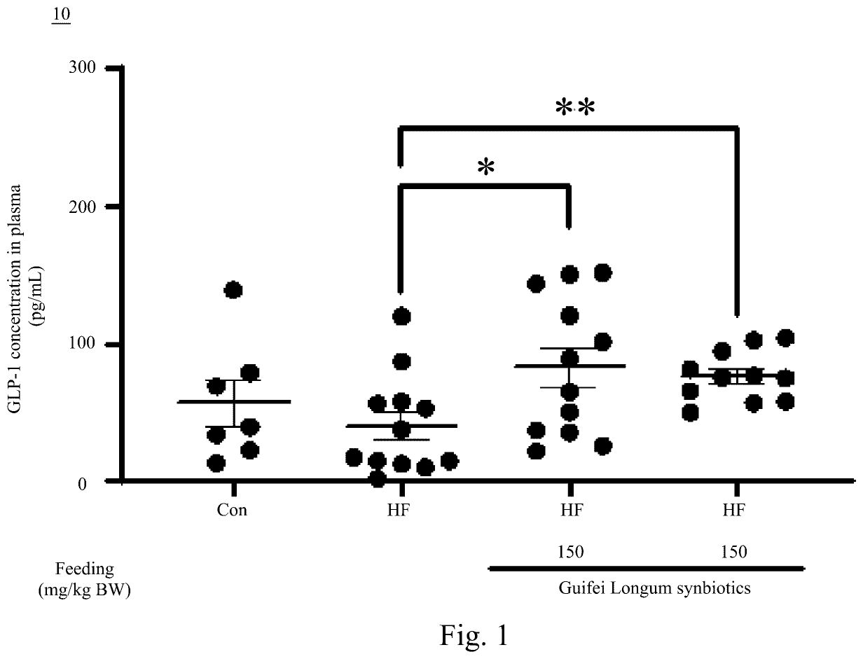

The results are shown in FIG. 1 drawing 10. After feeding mice with different samples for 8 weeks, the differences in blood GLP-1 secretion of mice in different groups after taking glucose were tested. The blood GLP-1 concentration of the control group was 56.63 pg/mL, the blood GLP-1 concentration of obese mice was 40.32 pg/mL, the blood GLP-1 concentration of obese mice taking low-dose Guifei Longum synbiotics was 82.82 pg/mL, and the blood GLP-1 concentration of obese mice taking high-dose Guifei Longum synbiotics was 76.48 pg/mL. According to statistical analysis, compared with the obese mice group, the blood GLP-1 of the obese mice taking low-dose or high-dose Guifei Longum synbiotics groups was significantly increased (unpaired t-test, *:p<0.05, **:p<0.01).

### EXAMPLE 2 GUIFEI LONGUM SYNBIOTICS INCREASE THE AMOUNT OF AKK BACTERIA IN THE BODY

This experiment used 8-week-old C57BL/6 female mice, which were randomly divided into an experimental group and a control group, with 8 mice in each group. At the beginning of the experiment, the experimental group was fed with Guifei Longum synbiotics (dose: 150 mg/kg BW) dissolved in 2.5% glycerol PBS; the control group was fed with an equal volume of PBS containing 2.5% glycerol. After 6 weeks of continuous tube feeding, feces were collected and quantitative analysis of AKK bacteria was performed.

In order to determine the amount of AKK bacteria in mouse feces, fresh mouse feces were collected at the 6th week after the beginning of the experiment, and DNA was isolated from the feces using a DNA extraction kit. The amount of AKK bacteria was then measured by real-time quantitative polymerase chain reaction (RT-PCR). The primer pair sequences designed for the Akkermansia muciniphila ATCC BAA-835 strain were: forward primer SEQ ID NO: 1 (CAGCACGTGAAGGTGGGGAC) and reverse primer SEQ ID NO: 2 (CCTTGCGGTTGGCTTCAGAT).

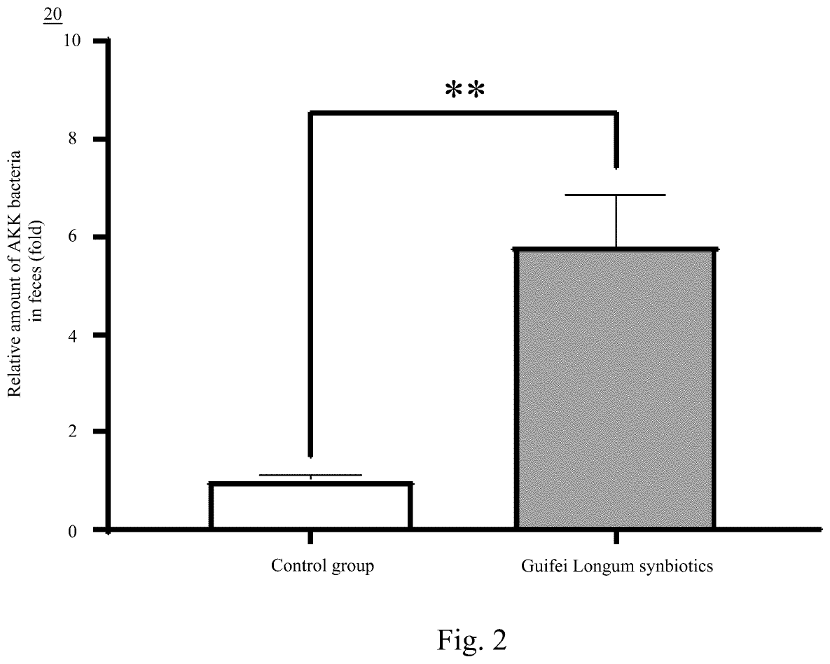

The results were shown in FIG. 2 drawing 20. After two groups of mice were fed PBS or Guifei Longum synbiotics for 6 weeks, fresh mouse feces were collected and the amount of AKK bacteria was measured. After statistical analysis, the relative amount of the control group was 1, and the relative average amount of the experimental group was 5.79. It can be seen that the amount of AKK bacteria in the mice of the experimental group was significantly higher than that of the control group (unpaired T test, **:p<0.01).

### EXAMPLE 3 GUIFEI LONGUM SYNBIOTICS INCREASE GLP-1 LEVELS IN HUMAN PLASMA

A total of 11 subjects were invited to this experiment, aged between 28 and 63 years old, 1 physiological male and 10 physiological females, with an average body fat rate of 33.89±4.24%, belonging to the obese group (normal is <25% for men and <30% for women). The experimental period was 5 weeks, during which the subjects will be asked to take Guifei Longum synbiotics capsules (500 mg/capsule) daily, twice a day, two capsules each time.

After humans ingest carbohydrates, fats, and proteins, intestinal L-cells will be induced to secrete GLP-1 into the blood. In order to test the difference in the efficacy of Guifei Longum synbiotics in promoting GLP-1 secretion in the human body, blood samples were first collected from subjects who had fasted for more than 8 hours. Then, liquid nutritional supplements were given to the subjects, and blood samples were collected again 30 minutes after they ingested. After plasma was separated from the blood samples, the amount of GLP-1 in the samples was analyzed using an ELISA kit. The nutritional supplements (commercially available Protison™-Cancer Nutrition Shake Oatmeal, SMAD Biotechnology CO., LTD.) ingested by the subjects had a calorie content of 372 kcal, a protein content of 21.6 grams, a fat content of 10.4 grams, and a carbohydrate content of 49.5 grams.

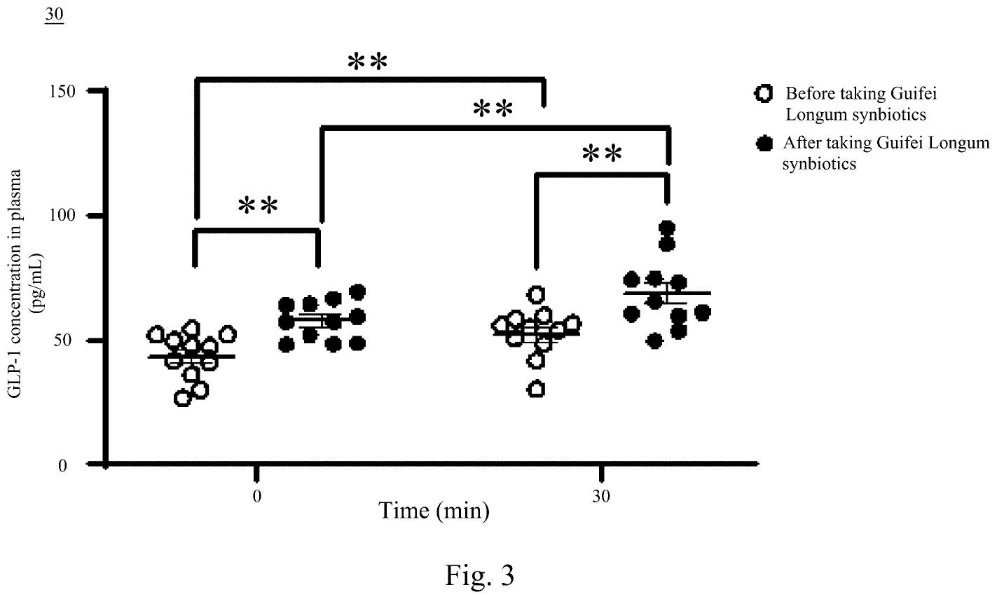

The results were shown in FIG. 3 drawing 30. When the subjects did not use Guifei Longum synbiotics (week 0) and were in a fasting state, the GLP-1 in the blood was 43.43 pg/mL; 30 minutes after taking the liquid nutritional supplement, the GLP-1 in the blood was 52.46 pg/mL. After using Guifei Longum synbiotics for 5 weeks and in a fasting state, the GLP-1 in the blood was 57.80 pg/mL; 30 minutes after taking the liquid nutritional supplement, the GLP-1 in the blood was 68.78 pg/mL. It can be seen that regardless of whether the subjects used Guifei Longum synbiotics or not, the blood GLP-1 after taking liquid nutritional supplements was higher than the fasting blood (paired T test, **:p<0.01). After using Guifei Longum synbiotics for 5 weeks, the blood GLP-1 concentrations at fasting or 30 minutes after taking liquid nutritional supplements were significantly higher than those at week 0 (paired T test, **:p<0.01).

In some embodiments of the present disclosure, it discloses that synbiotics have the effect of increasing glucagon-like peptide-1, and after administration of synbiotics, whether fasting or after meal, glucagon-like peptide-1 can be increased.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of increasing glucagon-like peptide-1, comprising administering to a subject in need thereof an effective amount of synbiotic composition comprising:
lychee polyphenols;
probiotics, wherein the probiotics comprises Bifidobacterium animalis BCRC910645, Bifidobacterium longum BCRC910812, or a combination thereof; and
methionine.

2. The method of claim 1, wherein the lychee polyphenols comprises proanthocyanidins, epicatechin, or a combination thereof.

3. The method of claim 1, wherein the synbiotic composition comprises:
a weight percentage of the lychee polyphenols from 1% to 30%,
a weight percentage of the probiotics from 10% to 50%, and
a weight percentage of the methionine from 0.5% to 20%,
based on a total weight of the synbiotic composition.

4. The method of claim 1, wherein the synbiotic composition further comprises indigestible maltodextrin, fructooligosaccharide, isomalto-oligosaccharide, probiotic fermented powder, rice bran concentrate, or a combination thereof.

5. The method of claim 4, wherein the probiotic fermented powder is prepared by the following:
culturing a combination of Lactobacillus salivarius subsp. salicinius AP-32 strain, Lactobacillus plantarum LPL28 strain, Lactobacillus acidophilus TYCA06 strain, and Bifidobacterium longum subsp. infantis BLI-02 strain in a culture medium comprising milk, milk powder, casein, soybean, bean product, or whey for fermentation;
after centrifuging the culture medium to obtain a fermentation liquid, removing strain activity of the fermentation liquid by heat sterilization, removing the strains in the fermentation liquid, and drying the fermentation liquid to form a dry powder; and
mixing the dry powder and maltodextrin to obtain the probiotic fermented powder.

6. The method of claim 4, wherein the rice bran concentrate is obtained by following:
mixing and grinding rice bran and water in a weight ratio of 1:1 to 1:10 to obtain a ground mixture;
hydrolyzing the ground mixture with amylase to obtain a hydrolyzed mixture; and
heating, centrifuging, and concentrating the hydrolyzed mixture to obtain the rice bran concentrate.

7. The method of claim 1, wherein the probiotics comprises the Bifidobacterium animalis BCRC910645 and the Bifidobacterium longum BCRC910812.

8. The method of claim 1, wherein the probiotics are live bacteria.

9. The method of claim 1, wherein an amount of the probiotics is from 1×10⁹ CFU/g to 1×10¹¹ CFU/g.

10. The method of claim 1, wherein the synbiotic composition is suitable for use in an obese subject.

11. The method of claim 10, wherein the obese subject is caused by following selected from the group consisting of diet, type 2 diabetes, hyperglycemia, glucose intolerance, dyslipidemia, insulin resistance, hyperinsulinemia, fatty liver, cardiovascular disease, stroke, cancer and combinations thereof.

12. The method of claim 1, wherein the synbiotic composition is a food composition or a pharmaceutical composition.

13. The method of claim 12, wherein the synbiotic composition is taken orally with a dose of from 1000 mg to 2400 mg per day.

14. The method of claim 12, wherein the synbiotic composition is taken orally with a dose of from 500 mg to 1200 mg twice per day.

15. The method of claim 1, wherein the synbiotic composition is administered orally or parenterally.

16. The method of claim 1, further comprising increasing an amount of Akkermansia muciniphila in intestine.

17. A method of preventing or treating glucagon-like peptide-1 deficiency, comprising administering to a subject in need thereof an effective amount of synbiotic composition comprising:
lychee polyphenols;
probiotics, wherein the probiotics comprises Bifidobacterium animalis BCRC910645, Bifidobacterium longum BCRC910812, or a combination thereof; and
methionine.

18. The method of claim 17, wherein the lychee polyphenols comprises proanthocyanidins, epicatechin, or a combination thereof.

19. The method of claim 17, wherein the synbiotic composition comprises:
a weight percentage of the lychee polyphenols from 1% to 30%,
a weight percentage of the probiotics from 10% to 50%, and
a weight percentage of the methionine from 0.5% to 20%,
based on a total weight of the synbiotic composition.

20. The method of claim 17, wherein the synbiotic composition further comprises indigestible maltodextrin, fructooligosaccharide, isomalto-oligosaccharide, probiotic fermented powder, rice bran concentrate, or a combination thereof.


SEQUENCE LISTING (2 sequences)

SEQ ID NO: 1 - DNA, 20 bp, synthetic construct
  source 1..20: mol_type = other DNA
cagcacgtgaaggtggggac

SEQ ID NO: 2 - DNA, 20 bp, synthetic construct
  source 1..20: mol_type = other DNA
ccttgcggttggcttcagat